(19) United States
(12) Patent Application Publication
(10) Pub. No.: US 2019/0365289 A1
(43) Pub. Date: Dec. 5, 2019

(54) ESTIMATING POWER USAGE BASED ON A TRAJECTORY

(71) Applicant: NTT Innovation Institute, Inc., East Palo Alto, CA (US)

(72) Inventor: Yasuyuki Kataoka, Sunnyvale, CA (US)

(21) Appl. No.: 15/994,566

(22) Filed: May 31, 2018

(51) Int. Cl. A61B 5/11; G06N 3/08; G01S 19/19

(52) U.S. Cl. CPC A61B 5/1112; A61B 2505/09; G01S 19/19; G06N 3/08

(57) ABSTRACT

A method for estimating power usage of a body in motion may include accessing location data associated with the body in motion, determining a trajectory of the body in motion based on the location data, encoding the trajectory, and estimating the power usage based on the encoded trajectory. The power usage may further be estimated based on one or more programmed features, which may include one or more of friction, wind resistance, potential energy, or kinetic energy. The method may further include estimating a second power usage of one or more second bodies in motion and estimating a competitive outcome based on the power usage and the second power usage.

### TECHNICAL FIELD

Embodiments of the present disclosure relate generally to estimating the power usage of a body in motion and more particularly to estimating power usage based on a trajectory.

### BACKGROUND

Understanding how power is used to propel a body in motion (e.g., to move people and objects) is useful in a variety of applications. For example, in the case of human- or animal-powered motion (e.g., walking, running, galloping, bicycling, skating, etc.), tracking power usage may be used for applications such as improving the performance of athletes, assisting in the rehabilitation of medical patients, studying the behavior of animals, and/or the like. Similarly, in the case of mechanically powered motion (e.g., the propulsion of cars, boats, airplanes, etc.), tracking power usage may be used for purposes including enhancing the speed and fuel efficiency of vehicles.

Accordingly, it is desirable to develop improved techniques for tracking the power usage of a body in motion.

### BRIEF DESCRIPTION OF THE DRAWINGS

FIG. 1 is a simplified diagram of a system for estimating power usage according to some embodiments.

FIG. 2 is a simplified diagram of a model for estimating power usage according to some embodiments.

FIG. 3 is a simplified diagram of a trajectory encoder according to some embodiments.

FIG. 4 is a simplified diagram of a competitive outcome prediction model according to some embodiments.

FIG. 5 is a simplified diagram of a training configuration for a neural network model according to some embodiments.

FIG. 6 is a simplified diagram of a method for estimating power usage according to some embodiments.

FIG. 7 is a simplified diagram of a method for training a neural network model according to some embodiments.

FIGS. 8A-8D are simplified diagrams of an experimental evaluation of a model for predicting power usage according to some embodiments.

### DETAILED DESCRIPTION

Competitive athletes, such as racers (e.g., bicycle racers, speed skaters, marathon runners, etc.), are an illustrative group of individuals that may generally be interested in tracking power usage. Parties that may seek to monitor power usage information include the athletes themselves, their coaches, and other members of the athletic team. For example, muscle power usage, possibly in combination with other physiological information (e.g., heart rate information), may be used to develop improved training regimens and racing strategies. Similarly, fans and other enthusiasts of competitive racing may seek deeper engagement in the sport by monitoring power usage information. For example, fans and recreational athletes may enjoy learning how top racers conserve or exert energy at various points in a race.

Before, during, and after competitions, competitive athletes may employ various strategies and tactics based on muscle fatigue. During a bicycle race, for instance, riders are often concerned with the timing of breakaways, e.g., when a sprinter or group of riders should break away from the peloton to maximize the likelihood of success. Whether or not the peloton is able to catch the breakaway group may hinge on the relative muscle fatigue of the racers in each group. Accordingly, riders, coaches, observers, and others may be interested in monitoring the muscle power usage of the racers throughout the race to evaluate the likelihood that the peloton will catch a given breakaway group.

In some cases, power usage may be measured directly using a power meter. For example, a bicycle (and other types of user-powered vehicles) may be equipped with a power meter that directly measures the power exerted on the pedals by the rider. However, dedicated power meters may be prohibitively bulky or expensive for some applications. For example, professional bicycle riders may prefer not to use dedicated power meters out of a desire to reduce the weight of their bicycles as much as possible during a competition. Meanwhile, recreational bicycle riders may be wary of the costs of dedicated power monitoring equipment. Moreover, access to data from dedicated power meters may be restricted, for example, to the rider's team members. Consequently, other interested parties (e.g., racing fans among the general public) may not have access to the power usage information from dedicated power meters.

Accordingly, it is desirable to develop improved techniques for monitoring power usage. In particular, it is desirable to develop techniques to predict power usage based on available data (e.g., publicly available location tracking data), rather than or in addition to directly measuring power usage using a dedicated power meter.

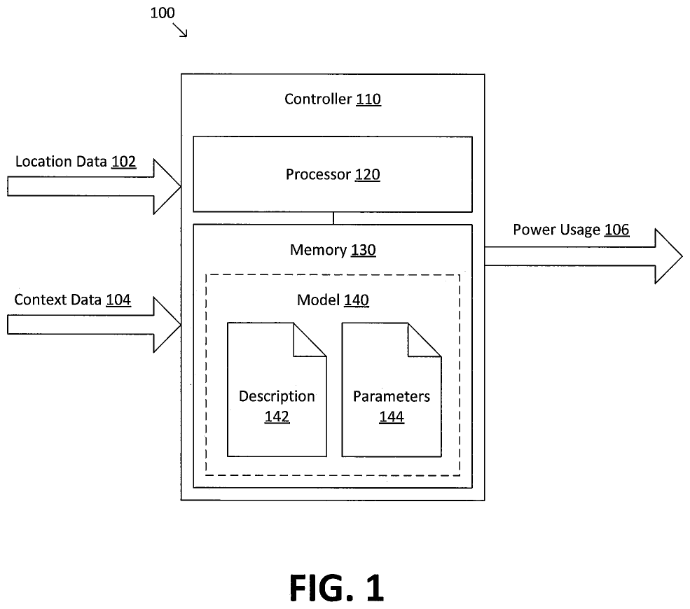

FIG. 1 is a simplified diagram of a system 100 for estimating the power usage of a body in motion according to some embodiments. According to some embodiments, system 100 may receive location data 102 and context data 104 and estimate power usage 106 based on location data 102 and context data 104. System 100 may estimate power usage in real-time (e.g., during a race) based on real-time data feeds, or at a later time based on stored data.

Location data 102 generally corresponds to data received from a location tracker associated with the body in motion. For example, location data 102 may be received from a wearable location tracker worn by a person (e.g., a walker, a runner, a bicycle rider, an ice skater, etc.) or an animal. Similarly, location data 102 may be received from a location tracker mounted to or included in an object or vehicle, such as a location tracker attached to a bicycle seat or a bicycle frame. In some embodiments, location data 102 may correspond to GPS data received from the location tracker. Additionally or alternately, location data 102 may be determined from image or video data, e.g., from a video camera capturing video from a race track, or from other suitable means of determining the location of a body in motion. In some embodiments, location data 102 may be retrieved from a publicly available source, e.g., from an Internet or television broadcaster of an athletic event.

Context data 104 generally corresponds to additional information associated with the person or object in motion that may be used to estimate the power usage. For example, context data 104 may include the wind speed and direction at the location of the body in motion, the altitude and slope at the location, the position of a racer relative to other racers in a cluster, the velocity of the body in motion (which may be derived from location data 102), and/or the like. Such data may be received from a variety of sources, including wind sensors, remote or local databases (e.g., digital maps), and/or the like.

As depicted in FIG. 1, system 100 includes a controller 110. In some embodiments, controller 110 may include a processor 120 (e.g., one or more hardware processors). Although processor 120 may include one or more general purpose central processing units (CPUs), processor 120 may additionally or alternately include at least one processor that provides accelerated performance when evaluating neural network models. For example, processor 120 may include a graphics processing unit (GPU), an application specific integrated circuit (ASIC), a field programmable gate array (FPGA), a tensor processing unit (TPU), a digital signal processor (DSP), a single-instruction multiple-data (SIMD) processor, and/or the like. Generally, such processors may accelerate various computing tasks associated with evaluating neural network models (e.g., training, prediction, preprocessing, and/or the like) by an order of magnitude or more in comparison to a general purpose CPU.

Controller 110 may further include a memory 130 (e.g., one or more non-transitory memories). Memory 130 may include various types of short-term and/or long-term storage modules including cache memory, static random access memory (SRAM), dynamic random access memory (DRAM), non-volatile memory (NVM), flash memory, solid state drives (SSD), hard disk drives (HDD), optical storage media, magnetic tape, and/or the like. In some embodiments, memory 130 may store instructions that are executable by processor 120 to cause processor 120 to perform operations corresponding to processes disclosed herein and described in more detail below.

Processor 120 and/or memory 130 may be arranged in any suitable physical arrangement. In some embodiments, processor 120 and/or memory 130 may be implemented on a same board, in a same package (e.g., system-in-package), on a same chip (e.g., system-on-chip), and/or the like. In some embodiments, processor 120 and/or memory 130 may include distributed, virtualized, and/or containerized computing resources. Consistent with such embodiments, processor 120 and/or memory 130 (or portions thereof) may be located in one or more data centers and/or cloud computing facilities. Additionally or alternately, processor 120 and/or memory 130 (or portions thereof) may be implemented on a mobile device, such as mobile device of the person or object in motion (e.g., a bicycle rider).

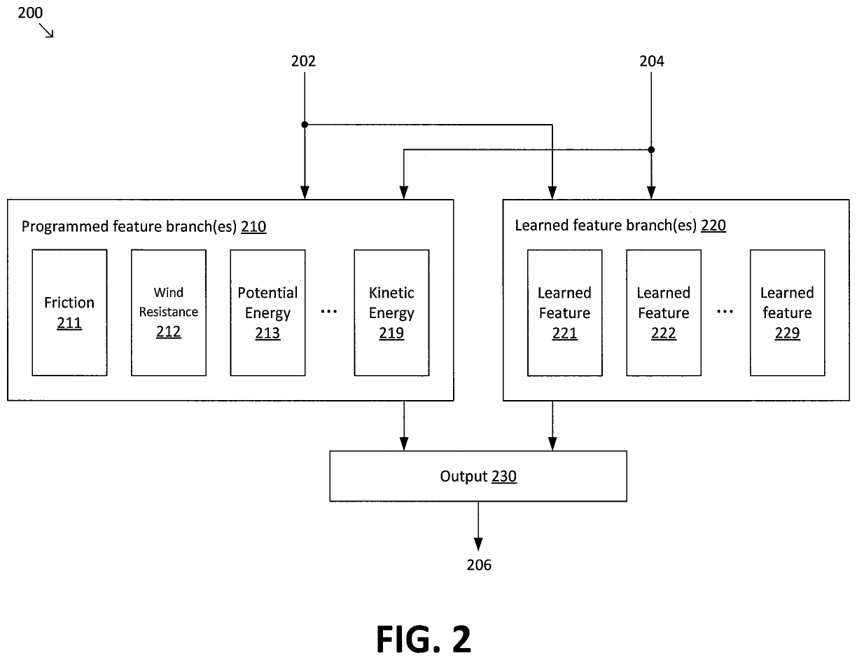

In some embodiments, memory 130 may store a model 140 that is evaluated by processor 120 during the estimation of power usage 106. Model 140 may include a plurality of neural network layers. Examples of neural network layers include densely connected layers, convolutional layers, recurrent layers, pooling layers, dropout layers, and/or the like. In some embodiments, model 140 may include at least one hidden layer that is not directly connected to either an input or an output of the neural network. Model 140 may further include a plurality of model parameters (e.g., weights and/or biases) that are learned according to a machine learning process. Examples of machine learning processes include supervised learning, reinforcement learning, unsupervised learning, and/or the like. Embodiments of model 140 are described in further detail below with reference to FIGS. 2-7.

Model 140 may be stored in memory 130 using any number of files and/or data structures. As depicted in FIG. 1, model 140 includes a model description file 142 that defines a computational graph of model 140 (e.g., a sequence of neural network layers) and a model parameters file 144 that stores parameters of model 140 (e.g., weights and/or biases). In general, model description file 142 and/or model parameters file 144 may store information associated with model 140 in any suitable format, including but not limited to structured, unstructured, serialized, and/or database formats.

FIG. 2 is a simplified diagram of a model 200 for estimating power usage of a body in motion according to some embodiments. According to some embodiments consistent with FIG. 1, model 200 may be used to implement model 140. In some embodiments, model 200 may receive location data 202 and context data 204 and estimate power usage 206 based on the received data. In embodiments consistent with FIG. 1, location data 202, context data 204, and power usage 206 may generally correspond to location data 102, context data 104, and power usage 106, respectively.

Model 200 may include one or more programmed feature branches 210 that evaluate one or more programmed features 211-219 based on location data 202 and/or context data 204. In some embodiments, programmed features 211-219 may evaluate features known to be correlated with power usage 206 based on a priori knowledge, e.g., based on physics models. FIG. 2 illustrates four examples of programmed features 211-219: (1) friction 211, which corresponds to the frictional force exerted upon the body in motion, (2) wind resistance 212, which corresponds to the wind resistance against the body in motion, (3) potential energy 213, which corresponds to the potential energy of the body in motion, and (4) kinetic energy 219, which corresponds to the kinetic energy of the body in motion.

In some embodiments, friction 211 may be evaluated based on the following equation:

P_{f}=C_{f}v_{b}mg (Eq. 1)

Where C_{f} denotes a friction coefficient, v_{b} denotes the velocity of the body in motion (e.g., a bicycle), m denotes the mass of the body in motion (e.g., the total mass of the bicycle and rider), and g denotes the standard gravity.

In some embodiments, wind resistance 212 may be evaluated based on the following equation:

P_{w}=½C_{d}Aρ(v_{b}−v_{w})² (Eq. 2)

Where C_{d} denotes a drag coefficient, A denotes the frontal cross-section area of the body in motion, ρ denotes the air density, and v_{w} denotes the wind velocity.

In some embodiments, potential energy 213 may be evaluated based on the following equation:

$${P}_{p} = mg \frac{Δ h}{Δ T} \qquad ( Eq . 3 )$$

Where ΔT denotes a duration of a sampling interval and Δh denotes the change in elevation of the body in motion between the beginning and end of the sampling interval.

In some embodiments, kinetic energy 219 may be evaluated based on the following equation:

$${P}_{w} = \frac{m}{2 Δ T} ( {v}_{n}^{2} - {v}_{p}^{2} ) \qquad ( Eq . 4 )$$

Where vᵥ denotes the velocity of the body in motion at the beginning of the sampling interval and vᵣ, denotes the velocity of the body in motion at the end of the sampling interval.

Although standard values for the physical constants used in equations 1-4 (e.g., the friction coefficient, drag coefficient, air density, etc.) are available, using the standard values in model 200 may result in low accuracy due to various non-idealities (e.g., sensor calibration). Accordingly, one or more of the physical constants in equations 1-4 may be trainable parameters that are learned according to a machine learning process.

It is to be understood that programmed features 211-219 are merely illustrative, and that various additional or alternative features may be included in model 200. In some embodiments, various programmed features 211-219 may be included or excluded from model 200 based on application-specific empirical correlations. For example, in bicycle racing, it is empirically observed that wind resistance 212 is a dominant factor in the computation of power usage. It is further observed that the wind resistance encountered by a rider may vary significantly from the measured wind velocity in the vicinity of the rider based on the rider's position in a cluster of riders. For example, a rider in the second row of a cluster may encounter half the wind resistance of the riders in the front of the cluster. Accordingly, the position of a rider in a cluster may be included among programmed features 211-219 to improve the accuracy of model 200 in bicycle racing applications.

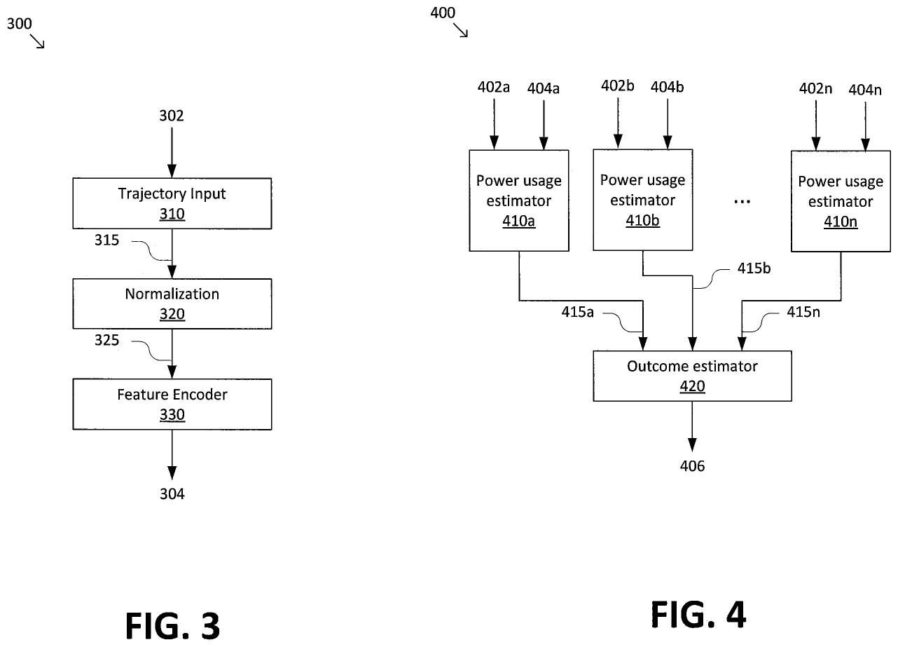

Model 200 may further include one or more learned feature branches 220 that evaluate one or more learned features 221-229 based on location data 202 and/or context data 204. Whereas programmed features 211-219 may generally be selected empirically or from physical models for power usage, learned features 221-229 correspond to feature representations that are determined by feature engineering. In this regard, learned features 221-229 may be evaluated using neural network models that are trained according to a machine learning process, as discussed in further detail below with reference to FIG. 3.

Model 200 may also include an output stage 230 that evaluates power usage 206 based on representations (e.g., scalar or vector representations) of programmed features 211-219 and learned features 221-229. In some embodiments, output stage 230 may be evaluated using a regression model and/or a neural network model. For example, output stage 230 may be implemented using a decision tree-based regression model such as the random forest regression model or the XGBoost regression model. Although a variety of regression models may be used, the use of a tree-based regression model offers several advantages, including a flexible number of trees, high accuracy in situations where relatively little training data is available, and an explanatory structure that allows interpretation of the results. Tree-based regression models may also be used for latency-sensitive applications of model 200, such as real-time applications (e.g., during a sports event), where the latency associated with evaluating model 200 may be an important consideration. To illustrate, it may be desirable to predict the real-time power usage of each rider in a 200-rider bicycle race once per second. Accordingly, each prediction should be evaluated with an average latency of five milliseconds or less, a budget that includes latency associated with retrieving location data 202 and/or context data 204 (e.g., database retrieval). In such applications, tree-based regression models may achieve sufficiently low latency to satisfy the constraints.

In some embodiments, output stage 230 may be implemented using a recurrent neural network model, such as deep learning models based on stacked long short-term memory (LSTM) layers and/or gated recurrent units (GRUs). The use of a recurrent neural network may improve the accuracy of output layer 230 by detecting temporal relationships that affect power usage 206.

According to some embodiments, model 200 may correspond to a computational graph, in which case programmed feature branches 210, learned feature branches 220, and/or output stage 230 may correspond to collections of nodes in the computational graph. Consistent with such embodiments, various representations used by model 200, such as representations corresponding to location data 202, context data 204, programmed features 211-219, and/or learned features 221-229, and/or any intermediate representations used by model 200, may correspond to real-valued tensors (e.g., scalars, vectors, multidimensional arrays, and/or the like) that are passed along edges of the computational graph. Moreover, each node of the computation graph may perform one or more tensor operations, e.g., transforming one or more input representations of the node into one or more output representations of the node. Examples of tensor operations performed at various nodes may include matrix multiplication, n-dimensional convolution, normalization, element-wise operations, and/or the like.

FIG. 3 is a simplified diagram of a trajectory encoder 300 according to some embodiments. According to some embodiments consistent with FIGS. 1-2, trajectory encoder 300 may be used to evaluate one or more of learned features 221-229. Consistent with such embodiments, trajectory encoder 300 may receive location data 302 and generate a trajectory representation 304 that is provided to output stage 230.

In general, trajectory encoder 300 may be used in applications where the trajectory of a body in motion is correlated with the power usage of the body in motion. For example, in the case of bicycle racing, a rider is likely to stop pedaling when making a sharp turn. Accordingly, when the bicycle rider follows a curved trajectory, the power usage is likely to be lower than when the rider follows a straight trajectory. In this regard, trajectory encoder 300 generates a representation (e.g., a vector representation) of the trajectory of a body in motion over a sampling interval, which may be used by output stage 230 to evaluate power usage 206.

Trajectory encoder 300 may include a trajectory input layer 310 that receives location data 302 and generates an input representation 315 of the trajectory. For example, input representation 315 may correspond to an image representation of the trajectory (e.g., a two or three dimensional binary image in which pixels along the trajectory are set to a first logical value (e.g., one) and other pixels are set to a second logical value (e.g., zero)), a coordinate representation of the trajectory (e.g., a set of coordinates in two or three dimensions that defines the trajectory), a functional representation of the trajectory (e.g., a set of parameters of a two or three dimensional function that defines the trajectory), and/or the like. Input representation 315 may have a fixed size, which may correspond to a portion of the trajectory having a fixed duration of time, a fixed distance of travel, a fixed number of samples, and/or the like. For example, input representation 315 may include a coordinate representation of the trajectory from t−NΔT to t+NΔT, where t is the current time, ΔT is the sampling interval, and N is the number of samples before and after the current time included in the trajectory. Consistent with such examples, the dimensionality of input representation 315 may be 3(2N+1), in a case where each sample includes three coordinates (e.g., an x, y, and z coordinate).

Although input representation 315 may be directly suitable for generating trajectory representation 304, it is observed that various symmetries (e.g., rotational symmetry, reflection symmetry, etc.) exist that may be used to reduce the sample space and improve training speed. For example, in the case of bicycle racing, power usage is likely to be similar regardless of the direction the rider is travelling (e.g., north, south, east, or west). Similarly, power usage is likely to be similar regardless of the direction the rider is turning (e.g., to the left or to the right). Conversely, power usage is likely to be different depending on whether the rider is travelling uphill or downhill.

Accordingly, trajectory encoder 300 may include a normalization layer 320 that applies one or more geometric transformations (e.g., rotations, reflections, displacements, etc.) to input representation 315 to generate a normalized representation 325 that accounts for one or more symmetries of input representation 315. In some embodiments, normalization layer 320 may apply rotation normalization based on a vector associated with the trajectory. For example, the vector may correspond to a displacement between two points along the trajectory separated by a fixed number of samples, an average direction of the trajectory, and/or the like. In this regard, normalization layer 320 may rotate input representation 315 such that the corresponding vector is oriented along a particular direction and axis (e.g., the positive direction of they axis). In some embodiments, normalization layer 320 may further apply one or more standardization operations, e.g., to center input representation 315 along one or more axes (e.g., the z axis).

Trajectory encoder 300 may further include a feature encoder layer 330 that generates trajectory representation 304 based on normalized representation 325. According to some embodiments, trajectory representation 304 may correspond to a compressed representation of normalized representation 325. For example, the compressed representation may achieve more than a three-fold reduction in dimensionality relative to normalized representation 325.

In some embodiments, trajectory encoder 300 may be implemented using an auto-encoder model, such as a de-noising auto-encoder, a deep auto-encoder, a stacked deep auto-encoder, and/or the like. The auto-encoder model may include a plurality of parameters that are learned according to a machine learning process. During training, a recovered version of the compressed representation is compared to an original input representation that was provided to trajectory encoder 300. In this manner, the plurality of parameters are trained such that that the compressed representation retains the features of the original representation, while reducing the dimensionalities. The depth of the auto-encoder model may be increased to achieve further compression.

FIG. 4 is a simplified diagram of a competitive outcome prediction model 400 according to some embodiments. In some embodiments, competitive outcome prediction model 400 may receive location data 402a-n and context data 404a-n corresponding to a plurality of competitors in a competition (e.g., cyclists in a bicycle race). Based on location data 402a-n and context data 404a-n, competitive outcome prediction model 400 may predict a competitive outcome 406 associated with the competition. In some embodiments, competitive outcome 406 may be predicted in real-time during the competition, providing competitors, coaches, and fans with insight into the current likelihood of a particular outcome, the efficacy of a particular strategy, and/or the like. For example, competitive outcome 406 may correspond to a predicted winner of the competition based on the muscle fatigue of the competitors. In the case of a bicycle race, competitive outcome 406 may correspond to the likelihood that the peloton will catch a breakaway group.

As depicted in FIG. 4, competitive outcome prediction model 400 includes a plurality of power usage estimators 410a-n corresponding to each of the plurality of competitors. Power usage estimators 410a-n generate respective estimates of power usage 415a-n for each of the competitors. In some embodiments consistent with FIGS. 1-3, power usage estimators 410a-n may be implemented using model 200. In some embodiments, power usage estimators 410a-n may be implemented a single instance of model 200 evaluated serially for each of the competitors, a plurality of instances of model 200 evaluated in parallel, or a combination thereof.

Competitive outcome prediction model 400 may further include an outcome estimator 420 that predicts competitive outcome 406 based on power usage 415a-n. In some embodiments, outcome estimator 420 may include a regression model, a neural network model, and/or the like. In addition to power usage 415a-n, outcome estimator 420 may receive other inputs to assist in the prediction of competitive outcome 406. For example, when predicting the likelihood that the peloton will catch the breakaway group, outcome estimator 420 may receive labels indicating which riders are in the peloton and which riders are in the breakaway group.

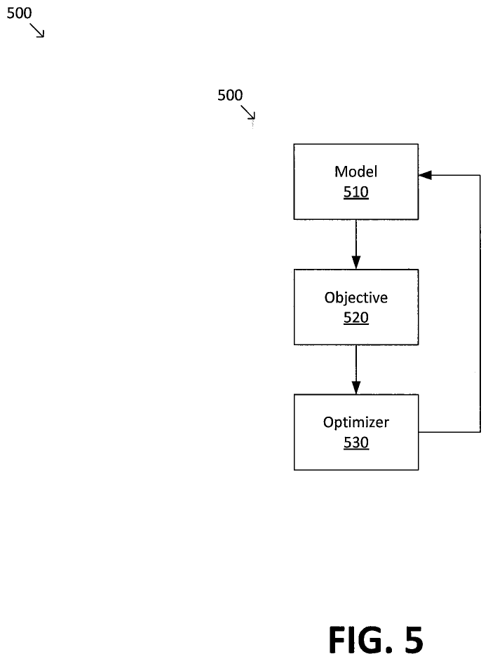

FIG. 5 is a simplified diagram of a training configuration 500 for a neural network model according to some embodiments. As depicted in FIG. 5, training configuration 500 is used to train a model 510. In some embodiments consistent with FIGS. 1-4, model 510 may be used to implement model 200. In some embodiments, model 510 may include a trajectory encoder, such as trajectory encoder 300.

According to some embodiments, training configuration 500 may be used to train a plurality of model parameters of model 510. During training, a large number of training examples (e.g., example sets of location data and context data) are provided to model 510. The output power usage predicted by model 510 is compared to a ground truth power usage for each of the training examples using a learning objective 520, which determines a loss and/or reward associated with a given prediction. In some embodiments, learning objective 520 may include a supervised learning objective, a reinforcement learning objective, and/or the like. In some embodiments, the ground truth power usage for a given training example may correspond to the power usage determined by a power meter, such as a bicycle power meter that directly measures the power exerted on the pedals by the rider.

The output of learning objective 520 (e.g., the loss and/or reward) is provided to an optimizer 530 to update the model parameters of model 510. For example, optimizer 530 may determine a gradient of the objective with respect to the model parameters and adjust the model parameters using back propagation. In some embodiments, optimizer 530 may include a gradient descent optimizer (e.g., stochastic gradient descent (SGD) optimizer), an ADAM optimizer, an Adagrad optimizer, an RMSprop optimizer, and/or the like. Various parameters may be supplied to optimizer 530 (e.g., a learning rate, a decay parameter, and/or the like) depending on the type of optimizer used.

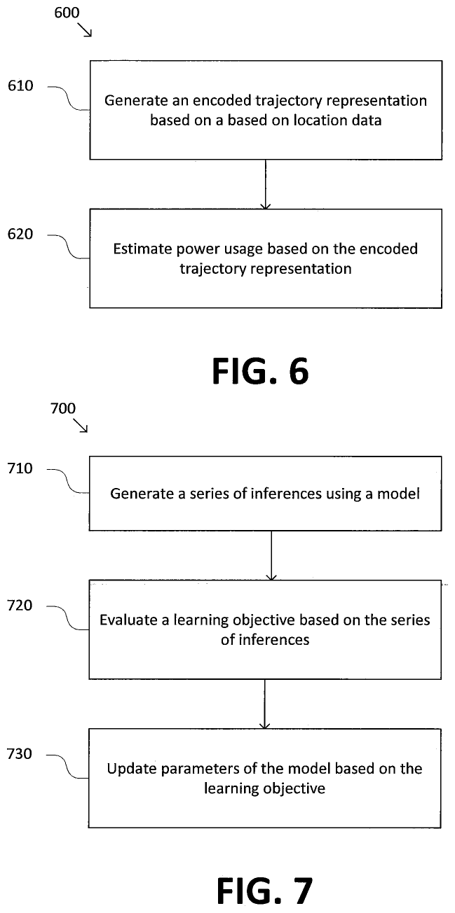

FIG. 6 is a simplified diagram of a method 600 for estimating power usage according to some embodiments. According to some embodiments consistent with FIGS. 1-5, method 600 may be performed using a processor, such as processor 120. In some embodiments, method 600 may be performed by evaluating a neural network model, such as model 140 and/or 200. In some embodiments, the neural network model may include a plurality of model parameters learned according to a machine learning process.

At a process 610, an encoded trajectory representation is generated based on location data. In some embodiments, the encoded trajectory representation may be generated by a trajectory encoder of the neural network model, such as trajectory encoder 300. In illustrative embodiments, the trajectory encoder may be implemented using an auto-encoder model, such as a de-noising auto-encoder, a deep auto-encoder, a stacked deep auto-encoder, and/or the like. In some embodiments the trajectory encoder may include a normalization layer that applies one or more geometric transformations to the location data, as described previously with respect to FIG. 3.

At a process 620, a power usage is estimated based on the trajectory representation. The power usage may further be estimated on one or more programmed features, such as friction, wind resistance, potential energy, kinetic energy, the position of a rider in a cluster of riders (in the case of a bicycle race or other type of race), and/or the like. In some embodiments, the power usage may be estimated using an output stage of the model, such as output stage 230. In some embodiments, the output stage may include a decision tree-based regression model, a recurrent neural network model, and/or the like. In QA applications, the inference may identify a span of text in the document that answers the question.

FIG. 7 is a simplified diagram of a method 700 for training a neural network model according to some embodiments. According to some embodiments consistent with FIGS. 1-6, method 700 may be used to train a neural network model, such as model 140 and/or 200, or a stage of a model, such as output stage 230 or trajectory encoder 300. During training, the model may be configured in a training configuration, such as training configuration 500. In some examples, method 700 may be performed iteratively over a large number of training examples to gradually train the neural network model.

At a process 710, a series of inferences is generated using the neural network model. In some embodiments, the series of inferences may be generated based on a training example, which may include location data and context data. In some embodiments, the series of inferences (e.g., estimated power usage) may be generated according to method 600.

At a process 720, a learning objective is evaluated based on the series of inferences. In some embodiments, the learning objective may correspond to learning objective 520. Consistent with such embodiments, the learning objective may include a supervised learning objective, a reinforcement learning objective, and/or the like.

At a process 730, the parameters of the neural network model are updated based on the learning objective. In some embodiments, the model parameters may be updated using an optimizer, such as optimizer 530. In some embodiments, the parameters may be updated by determining a gradient of the mixed learning objective with respect to the model parameters and updating the parameters based on the gradient.

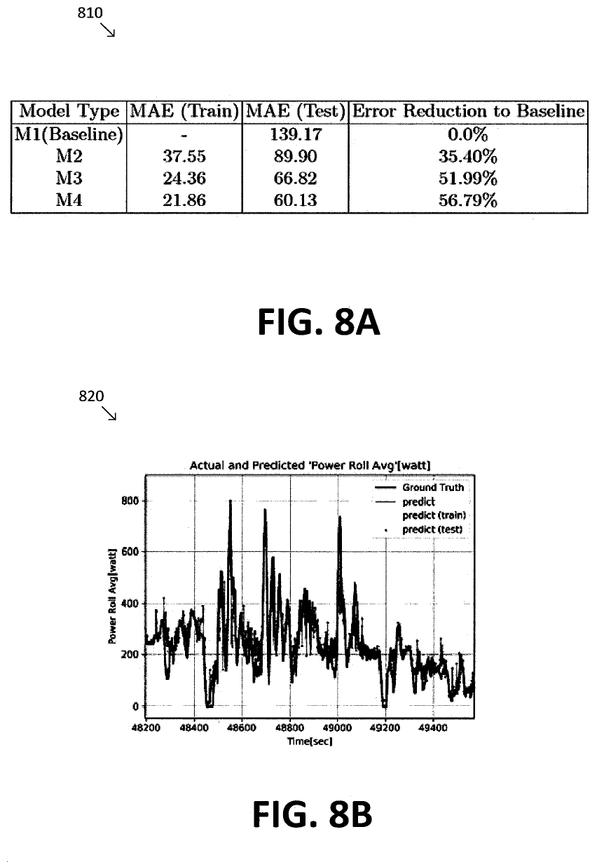

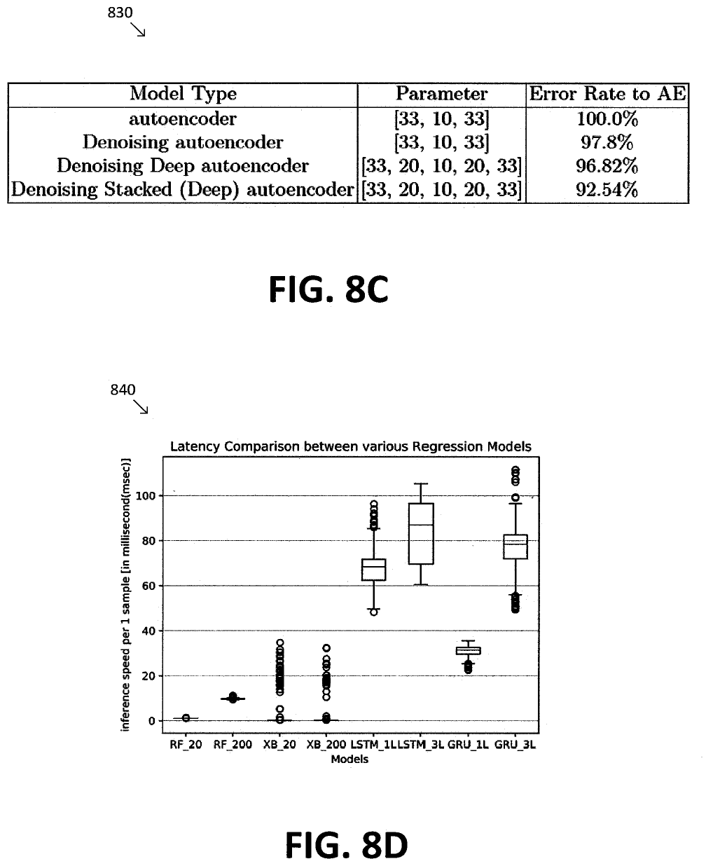

FIGS. 8A-8D are simplified diagrams of an experimental evaluation of a model for predicting power usage according to some embodiments. The model being evaluated trained and tested using data from professional bicycle riders at the 2017 Tour de France race.

FIG. 8A depicts a table 810 that compares the accuracy of four variants of the model: (first row) a baseline model that includes programmed features (e.g., programmed features 211-219) but does not include learned features (e.g., learned features 221-229); (second row) a model that uses the trajectory as a feature but does not include a trajectory encoder (e.g., trajectory encoder 300) that applies feature engineering to the trajectory, and does not include the programmed features; (third row) the model of the second row, except including the programmed features; and (fourth row) a model that includes a trajectory encoder as depicted in FIG. 3. The trajectory encoder is configured as a de-noising stacked auto-encoder with N=5 (i.e., the trajectory includes five samples before and after the current sample, making 11 samples total). The dimensionality of the auto-encoder is [33, 20, 10, 20, 33]. That is, the input layer has a dimensionality of 33 (three dimensions for each of the 11 samples), an intermediate layer has a dimensionality of 20, and the output layer has a dimensionality of 10; the last two layers are symmetrical to the first two layers and are used to recover the input representation during training. As indicated in the last column of table 810, the mean average error (MAE) during testing of the four models is reduced by over 50% relative to the baseline model (first row) when feature engineering is applied to the trajectory (last two rows).

FIG. 8B depicts a plot 820 that compares the power usage predicted using a model, such as model 200, to the ground truth power usage during a bicycle race. The ground truth power usage corresponds to the power usage (in watts) measured by a dedicated power meter attached to the bicycle. As indicated, the model predictions closely track the ground truth power usage over a wide range of power usages.

FIG. 8C depicts a table 830 comparing the error rate achieved using various types of auto-encoders in the trajectory encoder of the model. The parameter column denotes the dimensionality of each layer of the auto-encoder. As indicated, varying the type of auto-encoder (e.g., de-noising, deep, stacked, etc.) and the parameters of the auto-encoder (e.g., the presence or absence of an intermediate layer (with a dimensionality of 20) between the input layer (with a dimensionality of 33) and the output layer (with a dimensionality of 10)) may reduce the error rate relative to a standard auto-encoder (top row) by up to about 7.5%.

FIG. 8D depicts a plot 840 that compares the latency of various types of regression models used in the output stage of the model, such as output stage 240. From left to right, plot 840 compares (1) a random forest model with 20 trees, (2) a random forest model with 200 trees, (3) an XGBoost model with 20 trees, (4) an XGBoost model with 200 trees, (5) and LSTM model with one layer, (6) an LSTM model with three layers, (7) an GRU model with one layer, and (8) a GRU model with three layers. As indicated, the use of tree-based models (e.g., random forest and XGBoost) generally achieves lower latency than the recurrent neural network models (e.g., LSTM and GRU).

Although illustrative embodiments have been shown and described, a wide range of modifications, changes and substitutions are contemplated in the foregoing disclosure and in some instances, some features of the embodiments may be employed without a corresponding use of other features. One of ordinary skill in the art would recognize many variations, alternatives, and modifications. Thus, the scope of the present application should be limited only by the following claims, and it is appropriate that the claims be construed broadly and in a manner consistent with the scope of the embodiments disclosed herein.

What is claimed is:

1. A method for estimating power usage of a body in motion, comprising:
accessing location data associated with the body in motion;
determining a trajectory of the body in motion based on the location data;
encoding the trajectory; and
estimating the power usage based on the encoded trajectory.

2. The method of claim 1, further comprising normalizing the trajectory prior to encoding the trajectory.

3. The method of claim 2, wherein normalizing the trajectory comprises applying rotation normalization based on a vector associated with the trajectory.

4. The method of claim 3, wherein the vector corresponds to a displacement between two sampled points of the trajectory separated by a predetermined number of samples.

5. The method of claim 1, wherein encoding the trajectory comprises generating a compressed representation of the trajectory.

6. The method of claim 5, wherein the compressed representation is generated using an auto-encoder model.

7. The method of claim 6, wherein the auto-encoder model includes one or more of a de-noising auto-encoder, a deep auto-encoder, or a stacked deep auto-encoder.

8. The method of claim 1, wherein the power usage is further estimated based on one or more programmed features, the one or more programmed features being determined based on one or more of the location data or context data associated with the body in motion.

9. The method of claim 8, wherein the one or more programmed features include one or more of a friction, a wind resistance, a potential energy, or a kinetic energy.

10. The method of claim 1, further comprising:
estimating a second power usage of one or more second bodies in motion; and
estimating a competitive outcome based on the power usage and the second power usage.

11. The method of claim 10, wherein the competitive outcome corresponds to a likelihood that a peloton will catch a breakaway group in a racing competition.

12. The method of claim 1, wherein the power usage is estimated based on the encoded trajectory using one or more of a decision tree-based model or a recurrent neural network model.

13. A system for estimating power usage of a body in motion, comprising:
one or programmed feature branches configured to evaluate one or more programmed features based on one or more of location data and context data associated with the body in motion;
one or more learned feature branches configured to evaluate one or more learned features based on one or more of the location data and the context data; and
an output stage configured to predict the power usage based on the one or more programmed features and the one or more learned features.

14. The system of claim 13, wherein the one or more programmed features includes at least one of a friction, a wind resistance, a kinetic energy, or a potential energy.

15. The system of claim 13, wherein the one or more learned features includes a representation of a trajectory of the body in motion.

16. The system of claim 15, wherein the trajectory corresponds to a sequence of points comprising a predetermined number of samples from the location data.

17. The system of claim 15, wherein the representation corresponds to a compressed representation of the trajectory generated using an auto-encoder.

18. A non-transitory machine-readable medium having stored thereon machine-readable instructions executable to cause a system to perform operations comprising:
receiving, from a user-powered vehicle, location data associated with the user-powered vehicle;
determining a trajectory of the user-powered vehicle over a predetermined interval based on the location data;
encoding the trajectory using an auto-encoder model; and
estimating a power usage associated with the user-powered vehicle based on the encoded trajectory.

19. The non-transitory machine-readable medium of claim 18, wherein the power usage corresponds to a muscle fatigue of a user of the user-powered vehicle while performing an activity.

20. The non-transitory machine-readable medium of claim 18, wherein the user-powered vehicle does not include a dedicated power meter for tracking the power usage.
